(19) United States
(12) Patent Application Publication
(10) Pub. No.: US 2017/0216217 A1
(43) Pub. Date: Aug. 3, 2017

(54) SPHERICAL MICROCAPSULES WITH ENHANCED ORAL BIOAVAILABILITY

(71) Applicant: I2O Pharma Ltd., Ramat Gan (IL)

(72) Inventors: Michel Afargan, Ramat Gan (IL); Dan Gelvan, Ramat Gan (IL); Natalia Naraykin, Ramat Gan (IL)

(73) Assignee: I2O Pharma Ltd., Ramat Gan (IL)

(21) Appl. No.: 15/421,889

(22) Filed: Feb. 1, 2017

Related U.S. Application Data
(60) Provisional application No. 62/289,526, filed on Feb. 1, 2016.

(51) Int. Cl. A61K 9/50; A61K 47/26; A61K 38/03; A61K 9/51; A61K 31/513

(52) U.S. Cl. CPC A61K 9/5089; A61K 9/5026; A61K 9/5047; A61K 9/5192; A61K 9/5123; A61K 9/5153; A61K 31/513; A61K 38/03; A61K 47/26

(57) ABSTRACT

In one embodiment, the present invention provides a composition comprising a plurality of microcapsules, each comprising a shell and a core carrying an active agent selected from the group consisting of: (a) a non-hydrophilic active agent and (b) a hydrophilic active agent dissolved or suspended in an oil; wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

### CROSS-REFERENCE

This application claims priority to U.S. Provisional Patent Application Ser. No. 62/289,526, filed on Feb. 1, 2016, the entire content of which is incorporated by reference in its entirety.

The present invention relates to microcapsules for oral delivery.

### BACKGROUND

Oral administration of drugs is considered to be a convenient route of administration. However, certain drugs have poor bioavailability when administered orally. Encapsulation of drugs into microparticles can improve the bioavailability of drugs administered orally.

### SUMMARY

In one embodiment, the present invention provides a composition comprising a plurality of microcapsules, each comprising a shell and a core carrying an active agent selected from the group consisting of: (a) a non-hydrophilic active agent and (b) a hydrophilic active agent dissolved or suspended in an oil; wherein the shell comprises a polymeric coating, and wherein at least a portion of microcapsules in a sample of the composition are spherical when the sample of the microcapsules is viewed in a scanning electron microscope with a magnification in the range of between ×2000 and ×50000.

In one embodiment, the non-hydrophilic active agent is a lipophilic agent. In one embodiment, the non-hydrophilic active agent is lopinavir. In one embodiment, the active agent is a hydrophilic agent with a molecular weight of about 1 kiloDalton (kD). In one embodiment, the hydrophilic agent with a molecular weight of about 1 kiloDalton (kD) is octreotide. In one embodiment, the active agent is a hydrophilic agent with a molecular weight of about 4 kiloDalton (kD). In one embodiment, the hydrophilic agent with a molecular weight of about 4 kiloDalton (kD) is exenatide.

In one embodiment, the present invention provides a composition comprising a plurality of nanocapsules situated within a lumen formed by each shell of the plurality of microcapsules. In one embodiment, the plurality of nanocapsules are accommodated in the gel forming polymer, the plurality of nanocapsules each comprising a shell of polymeric coating and a liquid oil core comprising an active agent dissolved or suspended in the liquid oil core.

In one embodiment, the present invention provides a method of preparing a spherical microcapsules comprising a plurality of nanocapsules, the method comprises:
(a) spray drying sugar in a spray drying evaporator comprising a spray chamber and a cyclone, the amount of sugar being sufficiently in excess to cause at least a portion of the sugar to adhere to the surfaces of the evaporator;
(b) spray drying a dispersion comprising an active agent encapsulated in oil core droplets of the plurality of nanocapsules and a gel forming polymer or a combination of gel forming polymers to thereby obtain microcapsules, wherein the active agent is selected from the group consisting of: (a) a non-hydrophilic active agent and (b) a hydrophilic active agent dissolved or suspended in an oil;
wherein a shell of the oil core droplets comprises a polymeric coating, and; wherein at least a portion of the microcapsules are spherical when viewed in a scanning electron microscope with a magnification in the range of between ×2000 and ×50000.

In one embodiment, the present invention provides a method of preparing spherical microcapsules comprising a plurality of nanocapsules situated within a lumen formed by a shell of the spherical microcapsule, the nanocapsules comprising an oil core carrying an active agent and a polymeric shell coating, the method comprising: (a) spray drying sugar in a spray drying evaporator, the amount of sugar being sufficiently in excess to cause at least a portion of the sugar to adhere to the interior collection surfaces of the evaporator; (b) providing an organic phase comprising oil, a water miscible organic solvent, an active agent dissolved in the solvent and a polymer or combination of polymers for coating the oil core, wherein the active agent is selected from the group consisting of: (a) a non-hydrophilic active agent and (b) a hydrophilic active agent dissolved or suspended in an oil; (c) slowly adding water to the organic phase to obtain an emulsion; (d) continuously adding water to the emulsion to induce phase inversion of the emulsion thereby obtaining an oil in water (o/w) emulsion; (e) mixing the o/w emulsion with a gel forming polymer or a combination of gel forming polymers; and (f) removing the organic solvent by means of evaporation and water by means of spray drying to obtain the spherical microcapsules.

In one embodiment, the present invention provides a pharmaceutical dosage form comprising a composition and a physiologically acceptable carrier, wherein the composition comprises a plurality of microcapsules, each comprising a shell and a core carrying an active agent selected from the group consisting of: (a) a non-hydrophilic active agent and (b) a hydrophilic active agent dissolved or suspended in an oil; wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

In one embodiment, the present invention provides a method of increasing bioavailability of an active agent in a human subject's body; the method comprises administering to the human subject a composition or a pharmaceutical dosage form comprising the composition and a physiologically acceptable carrier, wherein the composition comprises a plurality of microcapsules, each comprising a shell and a core carrying an active agent selected from the group consisting of: (a) a non-hydrophilic active agent and (b) a hydrophilic active agent dissolved or suspended in an oil; wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

In one embodiment, the present invention provides a method of treating a subject for a pathological condition which requires for the treatment an effective blood level of an active agent, the method comprises administering to the subject a composition or a pharmaceutical dosage form comprising the composition and a physiologically acceptable carrier, wherein the composition comprises a plurality of microcapsules, each comprising a shell and a core carrying an active agent selected from the group consisting of: (a) a non-hydrophilic active agent and (b) a hydrophilic active agent dissolved or suspended in an oil; wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

### BRIEF DESCRIPTION OF THE DRAWINGS

FIG. 1 is a photograph of a small scale spray drying system according to some embodiments of the present invention, employing glass inner surfaces. The panel on the left of the figure is a close-up view of the nozzle. Reference number I refers to the beaker having a suspension of nanocapsules in the polymeric blend ready to feed into the nozzle (infused via the dashed line by a perilstatic pump), which is identified as Reference number III. Reference number II refers to the cyclone for microcapsule collection connected to the bottom of the collection flask. Reference number III refers to the nozzle where hot air heats the suspended feed and transforms the liquid to droplets transferred to the drying chamber. The sugar which is spray dried prior to the spray drying of the suspended polymeric blend adheres to the surface of the cyclone and the collection flask.

FIGS. 2A-2E shows a series of scanning electron microscope (SEM) images of microcapsules with irregular morphology containing either the lipophilic active agent lopinavir (FIGS. 2A-2D), or a hydrophilic synthetic peptide (FIG. 2E) made according to the methods described in Example 1, at various magnifications (×20000 (FIG. 2A), ×12000 (FIG. 2B), ×25000 (FIG. 2C) and ×50000 (FIG. 2D), ×3500 (FIG. 2E)).

FIG. 3 shows an SEM image of microcapsules with irregular morphology containing lopinavir, at a magnification of ×2000, made according to the methods described in Example 2, wherein a fine D-mannitol powder was added to the dry microcapsules following their formation and collected from the spray dryer, hence this D-mannitol was not spray dried.

FIGS. 4A-4C show SEM images of microcapsules containing a non-hydrophilic or a hydrophilic drug in comparison to placebo microcapsules made according to a method according to some embodiments of the present invention. FIG. 4A is an SEM image of symmetrical microcapsules bearing lopinavir loaded nanocapsules prepared according to some embodiments of the present invention, in which D-mannitol was spray-dried before the formation of the microcapsules. FIG. 4B is an SEM image of symmetrical microcapsules bearing peptide loaded nanocapsules prepared according to some embodiments of the present invention in which D-mannitol was spray-dried before the formation and collection of the microcapsules. FIG. 4C is an SEM image of symmetrical microcapsules bearing placebo nanocapsules without a drug prepared according to some embodiments of the present invention, in which D-mannitol was spray-dried before the formation and collection of the microcapsules.

FIG. 5 shows the pharmacokinetic profiles of microcapsules containing lopinavir made according to a method according to some embodiments of the present invention. Two batches of microcapsules without D-mannitol (B1 and B3) were compared to two batches of microcapsules that were produced after the inner surfaces of the evaporator were coated with dried D-mannitol according to some embodiments of the present invention (B4 and B7).

FIGS. 6A and 6B show the internal and external morphology of the microcapsules having a defined shell confirming a microcapsule structure; hence they are not solid microspheres. FIG. 6A shows nanocapsules released from microcapsules. FIGS. 6A and 6B confirm the nano-in-micro formulation having a plurality of nanocapsules entrapped in the inner space of a microcapsule.

FIG. 7 is an SEM image of mannitol particles as produced by a spray drying of mannitol solution 1% Wt in water. This sample is used as a control to distinguish between the morphology and size of mannitol particles in comparison to the microcapsules as depicted in FIGS. 4A, 4B and 4C. The mannitol sample of FIG. 7 was taken by utilizing a high resolution electron microscope, which is based on higher energy than the SEM used for FIGS. 4A-4C. The FIG. 7 shows the mannitol particles as solid microspheres having symmetrical morphology of size range from 1 to 10 μm; which is the same size range of the microcapsules. This microscopic image was produced under controlled scanning electron microscopy conditions of magnification ×3000. It was found that mannitol microspheres are not stable above this magnification, as they melt and loss their solid texture above a magnification of ×3000 while under the same conditions the microcapsules of the invention are stable. Hence, when a magnification above ×3000 is used only microcapsules are present in the sample. The relative instability of mannitol microspheres at a magnification above ×3000 enables differentiating between the symmetrical mannitol microspheres and the symmetrical microcapsules.

FIG. 8 is a high resolution SEM image ×2200 of a nanocapsule in microcapsule formulation of the hydrophilic polypeptide, exenatide. FIG. 8 depicts the symmetrical morphology of the exenatide nano-in-micro formulation as an example for hydrophilic macromolecules (polypeptide in comparison to the example of the small peptide of FIG. 2E) and support the efficacy of the mannitol procedure to improve the morphology of microcapsules for both small peptides and polypeptide with high molecular weight.

FIG. 9 is a high resolution SEM image of ×55000 of a single microcapsule and its surface morphology. In order to distinguish between the appearance of mannitol solid microspheres and the microcapsules in the SEM examples, a significant high energy is applied to a single microcapsule to confirm its integrity and stability. This example confirms the significant stability of the microcapsules in comparison to the low stability of the mannitol, which melts above the magnification energy of ×3000.

### DETAILED DESCRIPTION

For clarity of disclosure, and not by way of limitation, the detailed description of the invention is divided into the following subsections that describe or illustrate certain features, embodiments or applications of the present invention.

Further, as used herein, the term “comprising” is intended to mean that the microcapsules include the recited elements, but not excluding others. The term “consisting essentially of” is used to define microcapsules that include the recited elements but exclude other elements that may have an essential significance on the bioavailability of the active agent within a subject's body. “Consisting of” shall thus mean excluding more than trace elements of other elements. Embodiments defined by each of these transition terms are within the scope of this disclosure.

Further, all numerical values, e.g. when referring to the amounts or ranges of the elements constituting the microspheres, are approximations which are varied (+) or (−) by up to 20%, at times by up to 10% from the stated values. It is to be understood, even if not always explicitly stated, that all numerical designations are preceded by the term “about”.

### Definitions

As used herein, the term “accommodated” refers to enclosing, coating, embedding, surrounding, entrapping or any other manner of incorporating the active agent by the gel forming polymer(s) so as to provide a packed arrangement of the active agent with at least the gel protecting environment.

The term “at least a portion of the spherical microcapsules” as used therein refers to the portion that may be quantitatively determined, e.g. by viewing the image of the micrograph, as well as qualitatively determined, e.g. using image processing. “At least a portion” may refer to at least 5%, 10%, 20%, 30%, 40%, 50%, 60%, 70%, 80%, 90%, 95%, 98%, 99% or even 100% of the microcapsules being spherical at their outer surface.

The term “gel forming polymer” or “hydrogel forming polymer” refers to hydrophilic polymer which when wetted, forms a network of polymers that swell up or gels.

The term “nanocapsules” as used herein refers to nano- or subnano-scale structures comprising an oil droplet (fine oil drops) coated with a polymeric coating. The polymeric coating forms a hard shell enveloping and forming the oil core.

As used herein, the term “non-hydrophilic active agent” refers to any compound that is regarded as, at least to some extent, water repelling. In other words, any agent exhibiting low, medium or high hydrophobicity or lipophilicity would be regarded as a non-hydrophilic agent. A non-hydrophilic agent may be defined by parameters characterizing the partition/distribution coefficient of the agent (as a solute) between two phases for example, an organic solvent and water (the most commonly used system being octanol-water). Typically, a partition coefficient (log P) describes the hydrophobicity of neutral compounds, while the distribution coefficient (log D, being a combination of pKa and log P) is a measure of the pH dependent hydrophobicity of the agent. A non-hydrophilic active agent in accordance with the invention is any compound having a log P greater than 1.5.

As used herein, the term “plurality of microcapsules” refers to two or more microcapsules accommodated in the composition.

As used herein, the term “plurality of nanocapsules” refers to two or more nanocapsules accommodated in the gel-forming polymer.

As used herein, the term “shell” refers to any solid or semi-solid polymeric structure enclosing either, an oil droplet, or a spherical microcapsule.

The term “spherical microcapsules” as used herein, refers to micron- or submicron-scale particles which are typically composed of solid or semi-solid materials and capable of carrying and releasing a drug or any other active agent-containing nanocapsule enclosed therein. When referring to spherical morphology it should be understood as having its common meaning, i.e. a ball shaped, globular outer structure/shape/morphology.

The term “sugar” as used herein refers to a simple sugar, namely, mono, di or oligosaccharide or a sugar alcohol. Without being limited, examples of simple sugars that can be employed in the context of the present disclosure include inositole, lactose, glucose, fructose, pentose, mannose and sucrose and any isomer thereof.

The term “sugar alcohol” as used herein refers to a polyol, such as that formed from a saccharide. Without being limited thereto, a sugar alcohol may be any one of glycerol (3-carbon), erythritol (4-carbon), threitol (4-carbon), arabitol (5-carbon), xylitol (5-carbon), ribitol (5-carbon), mannitol (6-carbon), sorbitol (6-carbon), galactitol (6-carbon), fucitol (6-carbon), iditol (6-carbon, a cyclic sugar alcohol), volemitol (7-carbon), isomalt (12-carbon), maltitol (12-carbon), lactitol (12-carbon).

The term “water insoluble polymer” as used herein, refers to any polymer which does not lose more than 10% of its dry weight into an aqueous medium by dissolution, irrespective of the pH of the medium.

The term “water soluble polymer” as used herein, refers to any polymer which, when introduced into an aqueous phase at 25° C., at a mass concentration equal to 1%, make it possible to obtain a macroscopically homogeneous and transparent solution, i.e. a solution that has a minimum light transmittance value, at a wavelength equal to 500 nm, through a sample 0.1 cm thick, of at least 80%, or alternatively, of at least 90%.

### Preparation of Spherical Microcapsules

In some embodiments, the present invention provides spherical microcapsules comprising a plurality of nanocapsules situated within a lumen formed by the shell of the spherical microcapsule, the nanocapsules comprising an oil core carrying an active agent and a polymeric shell coating.

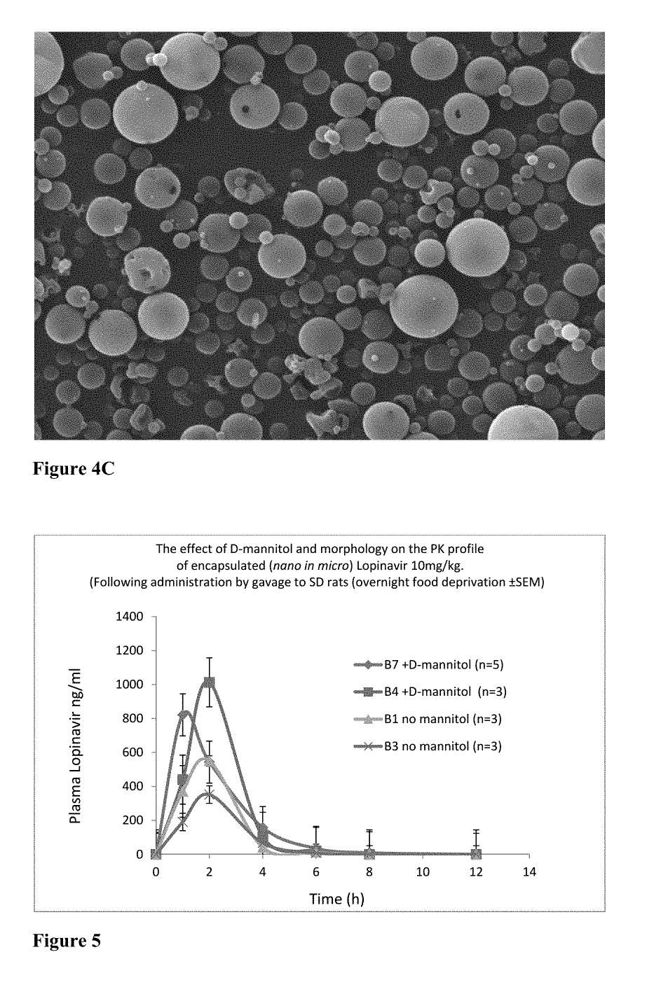

For illustrative purposes only, an example of a spherical microcapsule according to some embodiments of the present invention is shown in FIGS. 6A-6B. FIGS. 6A-6B show the internal and external morphology of the microcapsules having a defined shell confirming a microcapsule structure. FIGS. 6A and 6B confirm the nano-in-micro formulation, having a plurality of nanocapsules entrapped in the inner space of a microcapsule.

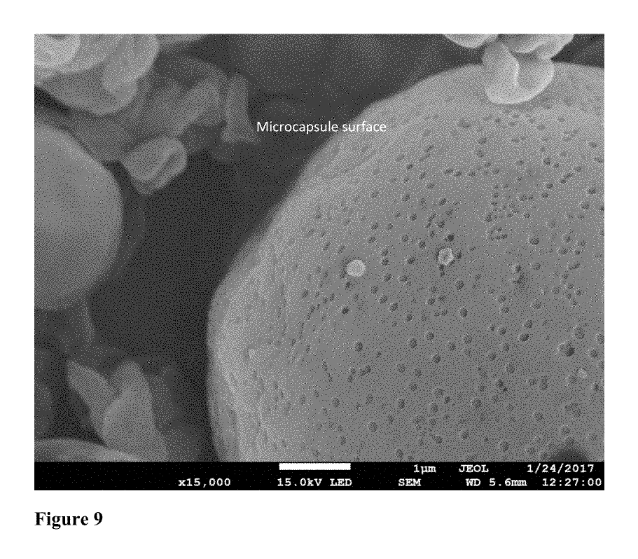

FIG. 9 is a high resolution SEM image of ×55000 of a single microcapsule and its surface morphology. Under high energy conditions, of ×15,000, no mannitol particles exist as such particles start to melt above 3,000. Therefore, such high energy conditions can distinguish between the mannitol and the microcapsules.

In some embodiments, the spherical microcapsules are defined as having a spherical symmetry where each point on the surface of the spherical microcapsule has essentially the same distance from the center.

In some embodiments, at least 5%, 10%, 20%, 30%, 40%, 50%, 60%, 70%, 80%, 90%, 95%, 98%, 99% or even 100% of the spherical microcapsules have a spherical symmetry.

In some embodiments, the present invention provides a method of preparing spherical microcapsules comprising a plurality of nanocapsules situated within a lumen formed by the shell of the spherical microcapsule, the nanocapsules comprising an oil core carrying an active agent and a polymeric shell coating, the method comprising:
(a) spray drying sugar in a spray drying evaporator, the amount of sugar being sufficiently in excess to cause at least a portion of the sugar to adhere to the interior collection surfaces of the evaporator;
(b) spray drying a dispersion comprising an active agent encapsulated in oil core droplets of the plurality of nanocapsules and a gel forming polymer or a combination of gel forming polymers to obtain microcapsules, wherein the active agent is selected from the group consisting of: (a) a non-hydrophilic active agent and (b) a hydrophilic active agent dissolved or suspended in an oil, wherein a shell of the oil core droplets comprises a polymeric coating;
wherein at least a portion of the microcapsules are spherical when viewed in a scanning electron microscope with a magnification in the range of between ×20000 and ×50000.

In some embodiments, the spray drying step (b) concentrates the sugar on the inner surfaces of the evaporator.

In some embodiments, the inner surfaces of the evaporator are glass. In some embodiments, the inner surfaces are stainless steel.

In some embodiments, the present invention provides a method of preparing spherical microcapsules comprising a plurality of nanocapsules situated within a lumen formed by the shell of the spherical microcapsule, the nanocapsules comprising an oil core carrying an active agent and a polymeric shell coating, the method comprising:
(a) spray drying sugar in a spray drying evaporator, the amount of sugar being sufficiently in excess to cause at least a portion of the sugar to adhere to the interior collection surfaces of the evaporator;
(b) providing an organic phase comprising oil, a water miscible organic solvent, an active agent dissolved in the solvent and a polymer or combination of polymers for coating the oil core, wherein the active agent is selected from the group consisting of: (a) a non-hydrophilic active agent and (b) a hydrophilic active agent suspended in an oil;
(c) slowly adding water to the organic phase to obtain an emulsion;
(d) continuously adding water to the emulsion to induce phase inversion of the emulsion thereby obtaining an oil in water (o/w) emulsion;
(e) mixing the o/w emulsion with a gel forming polymer or a combination of gel forming polymers;
(f) removing the organic solvent by means of evaporation and water by means of spray drying to obtain the spherical microcapsules.

The organic solvent used can be any organic solvent miscible with water that has a boiling point close to or lower than the boiling point of water. A non-limiting list of such organic solvents includes ethanol, methanol, acetone, ethyl acetate, isopropanol (bp 108° C., nonetheless regarded as volatile in the context of the present disclosure).

The use of a combination of oil and organic solvent enables the encapsulation within the nanocapsules of various active agents which can be essentially non-hydrophilic in nature, or capable of dissolving or being suspended in the oil core. The oil core can also include one or more non-hydrophilic excipients (e.g. lipophilic excipients). To this end, one or more excipients can be added in the organic phase. The excipient can be any excipient having at least 1% solubility in an oil phase. According to one embodiment, the excipient is a lipophilic surfactant, such as labrafil M 1944 CS, polysorbate 80, polysorbate 20.

Further, in accordance with this embodiment (where the agent is encapsulated in nanocapsules). Water is slowly added (i.e. added drop-wise) to the oil containing organic phase. At the beginning, an oil in water emulsion is formed, i.e. drops of water are suspended in the organic phase. However, continuous slow addition of water to the medium eventually results in an inverse phenomenon, where the continuous and non-continuous are ‘switched’ such that oil droplets coated with the polymer coating are suspended in water. The continuous slow addition of water is stopped upon achieving the inverse phenomenon (i.e. oil droplets coated with the polymer coating suspended in water).

When forming the nanocapsules, initially a water in oil (w/o) emulsion is formed and this w/o emulsion is converted to an oil in water (o/w) emulsion by the addition of water to the oil/organic phase.

The resulting emulsion comprising the oil droplets coated with the coating polymer(s) is then mixed with the solution of the gel forming polymer. Once the oil in water emulsion is formed and the gel forming polymer is added, the solvent (or mixture of solvents) and the water are essentially removed and the desired composition comprising the spherical microcapsules is obtained.

Spray drying is a mechanical microencapsulation method developed in the 1930s. Accordingly, an emulsion is atomized into a spray of droplets by pumping the slurry through a rotating disc into the heated compartment of a spray drying evaporator. There, the solvent as well as the water in the emulsion are evaporated to obtain the dry microcapsules.

The operation of the spray drying evaporator can vary depending on the type of device used. In some embodiments, the evaporator is operated under the following conditions:

Inlet temperature between 100° C. to 200° C., outlet temperature between 40° C. to 100° C. (the outlet temperature being at least 50° C. lower than the inlet temperature, aspiration of 60% to 100%, pump rate in the range of 10-40% that is 145-590 ml/h respectively and more specifically 20-30%, air flow in the range of 300-800 L/h and more specifically 600-700 L/h, and a nozzle cleaner range of 2-4. This is the in-process cleaning mode of the nozzle mode −2 means moderate rate of cleaning for spray drying of dispersions.

In some embodiments, the spray drying sugar comprises the step of dissolving the sugar in water, e.g. double distilled water (DDW) to obtain a solution of between 0.5%-10% w/v sugar, at times, between 0.5% to 5%, at times 2%±0.5%. The operating conditions of the spray drying of the sugar solution are identical to the spray drying of the spherical microcapsule dispersion despite the inlet and outlet temperatures of inlet 100-115° C. and outlet 45-60° C.

The existence of the dissolved sugar in the spray drying components is evidenced by its appearance as a dried solid particles/powder concentrated in the cyclone and the collection flask.

As noted, subsequent to spray drying of the sugar solution a dispersion of one or more gel forming polymers and the active agent is subjected to spray drying in the same spray drying system a priori “treated” with the sugar.

In some embodiments, the dispersion of the active agent with polymers is prepared by dissolving the active agent with surfactant in acetone followed by addition of the gel forming polymers in water at the desired concentration of components.

In some embodiments, the active agent is first encapsulated in nanostructures (e.g. nanocapsules).

In some embodiments, the composition comprises spherical microcapsules where the active agent is enclosed within nanocapsules. Such spherical microcapsules are spray dried as described in U.S. Pat. No. 9,023,386, the content of which is incorporated herein by reference, albeit in a spray dry evaporated a priori treated with the sugar, as described herein.

Without being limited by theory, during the flow within the spray drying system, most of the microcapsules are concentrated on the interior collection surfaces of the cyclone and the collecting flask and their relatively high affinity and surface area to the interior collection surfaces leads to considerable adsorption of the solid microcapsules to the glass, which in turn lead to the undesired irregular morphology of the microcapsules.

However, unexpectedly, when evaluating the effect on the morphology of microcapsules, with known additives such as D-mannitol known as a bulk agent (used to improve the dispersion of solid microparticles), it has been found that adding D-mannitol as a raw powder or fine powder to the microcapsule powder, did not affect the microcapsule and the bioavailability of this blend was not improved. Similarly, manipulations in the spray drying conditions, including changing the inlet and outlet temperature and the air flow, neither affected microcapsule morphology, nor bioavailability.

Spray-drying of D-mannitol solution before spray drying the material forming the microcapsules; unexpectedly resulted in change in morphology and improved bioavailability as further discussed below.

Without being bound by theory, this unexpected finding was postulated to obtain due to the adsorption of D-mannitol to the interior collection surfaces that create a barrier between the glass surface and the spray dried microcapsules, thereby reducing the surface tension and the adsorption of the microcapsules to the interior collection surfaces.

The spherical microcapsules according to some embodiments of the present invention are comprised of aggregates of nanocapsules incorporating (e.g. embedding, encapsulating, entrapping) the active agent. Typically, the average diameter of the spherical microcapsules according to some embodiments of the present invention, which is understood as weight-average diameter as determined by laser diffraction, ranges from approximately 10 μm to approximately 500 μm. In some embodiments, the average microsphere diameter is between about 10 μm and about 20 μm.

In some embodiments, the nanocapsules have an average diameter of between about 100 nm and about 1000 nm. In some embodiments, the nanocapsules have an average diameter of between about 100 nm to 900. In some embodiments, the nanocapsules have an average diameter of between about 100-300 nm to about 300-500 nm.

In some embodiments, the nanocapsules' size in a microsphere is essentially uniform with about 99% of the oil droplets having a diameter below 1 micron.

In some embodiments, the active agent is enclosed within the nanocapsule. As a result, in some embodiments, there is no direct contact between the active agent and the gel forming polymer forming the spherical microcapsules.

In some embodiments, upon wetting and swelling in the GI tract of the subject, the spherical microcapsules release the nanocapsule and not the “naked” active agent.

In some embodiments, the present invention provides pharmaceutical compositions comprising as the active component spherical microcapsules comprising a plurality of nanocapsules situated within a lumen formed by the shell of the spherical microcapsule, the nanocapsules comprising an oil core carrying an active agent and a polymeric shell coating.

In some embodiments, the oil core of the nanocapsules may comprise a single oil type or a combination of oils and can be selected from a wide range of usually usable oils from polar oils to non-polar oils, as long as they do not mix with the water phase and are a liquid as a whole in ambient and physiological temperatures, ranging from about 20° C. to about 37° C.

According to some embodiments, the oil droplets comprise an oil selected from long chain vegetable oils, ester oils, higher liquid alcohols, higher liquid fatty acids, natural fats and oils and silicone oils.

According to some embodiments, the oil core comprises a natural oil such as corn oil, peanut oil, coconut oil, castor oil, sesame oil, soybean oil, perilla oil, sunflower oil, argan oil and walnut oil.

In some embodiments, the oil droplets are each enclosed within a polymeric coating to form nanocapsules comprising the oil core and a polymeric shell surrounding the oil core.

In some embodiments, the polymeric coating provides a shell structure surrounding the oil core. In some embodiments, the shell can comprise a single polymer or a combination or blend of two or more polymers. When the polymeric coating comprises a blend of polymers, it is in accordance with some embodiments that at least one of the polymers is soluble at a pH above 5.0, or that at least one of the polymers is water soluble (pH independent).

In accordance with some embodiments, the combination of at least two polymers comprises a blend of polymers comprising a first polymer or polymers (group of polymers) which is either water soluble (pH independent) or soluble at a pH of above 5.0 and a second polymer or polymers (second group of polymers) which is water insoluble polymer.

The term “polymer soluble at a pH above 5.0” refers to any polymer that at a pH below 5.0 and at 25° C., it does not lose more than 10% of its dry weight into the medium by dissolution, while at the same temperature, in an aqueous medium having a pH above 5.0, it forms a hydrogel or dissolved to form a macroscopically homogeneous and transparent solution. Such polymers are referred to, at times; by the term “enteric polymers”.

Water soluble polymers suitable for use in some embodiments of the present invention include, but are not limited to, polyols and polycarbohydrates. Examples include hydroxylated celluloses, such as, for example, hydroxypropylmethyl cellulose and hydroxymethyl cellulose. Other suitable water soluble polymers include polyethylene glycol. Combinations of two or more water soluble polymers are also contemplated.

Non-limiting examples of enteric polymers suitable for use in some embodiments of the present invention include, but are not limited to hydroxypropylmethylcellulose phthalate (HP55), cellulose acetate phthalate, carboxy-methylcellulose phthalate, and any other cellulose phthalate derivative, shellac, poly(methacrylic acid-co-ethyl acrylate) 1:1 (Eudragit L100 55), zein.

In some embodiments, the enteric polymer is Eudragit L100 55.

Non-limiting examples of water insoluble polymers include cellulose esters such as di- and triacylates including mixed esters such as, for example, cellulose acetate, cellulose diacetate, cellulose triacetate, cellulose propionate, cellulose acetate butyrate, cellulose acetate propionate, cellulose tripropionate; cellulose ethers such as ethyl cellulose; nylons; polycarbonates; poly (dialkylsiloxanes); poly (methacrylic acid) esters; poly (acrylic acid) esters; poly (phenylene oxides); poly (vinyl alcohols); aromatic nitrogen-containing polymers; polymeric epoxides; regenerated cellulose; membrane-forming materials suitable for use in reverse osmosis or dialysis application; agar acetate; amylose triacetate; beta glucan acetate; acetaldehyde dimethyl acetate; cellulose acetate methyl carbamate; cellulose acetate succinate; cellulose acetate dimethylamino acetate; cellulose acetate ethyl carbonate; cellulose acetate chloroacetate; cellulose acetate ethyl oxalate; cellulose acetate propionate; poly (vinylmethylether) copolymers; cellulose acetate butyl sulfonate; cellulose acetate octate; cellulose acetate laurate; cellulose acetate p-toluene sulfonate; triacetate of locust gum bean; hydroxylated ethylene-vinyl acetate; cellulose acetate butyrate; wax or wax-like substances; fatty alcohols; hydrogenated vegetable oils; polyesters, homo and copolymer, such as polylactic acid or PLAGA and the like (for example poly(lactic-co-glycolic acid) PLGA), and combinations thereof.

In some embodiments, the water insoluble polymers are selected from the group consisting of: Poly(ethyl acrylate-co-methyl methacrylate-co-trimethylammonioethyl methacrylate chloride) 1:2:0.1 (Eudragit RS), Poly(ethyl acrylate-co-methyl methacrylate-co-trimethylammonioethyl methacrylate chloride) 1:2:0.2 (Eudragit RL), and combinations thereof.

In some embodiments, the nanocapsules comprise at least two polymers. In some embodiments, the first polymer is water insoluble polymer and the second polymer is soluble at a pH above about 5.0.

In accordance with some embodiments, the weight/weight ratio between the first polymer(s), i.e. the water insoluble polymer or group of polymers and the second group polymer(s), i.e. the polymer(s) soluble at pH above about 5.0 or group of such polymers is in the range between 5:95 and 50:50.

Without being bound by theory, it is believed that the ratio between the water insoluble polymer and the polymer soluble at pH above about 5.0 (the “non-insoluble” polymer) is important for controlling release of the active agent from the nanocapsules. Without being bound by theory, having a first polymer that is water insoluble and a second polymer that is soluble in water or soluble in water at a pH above 5.0 allows, following exposure of the nanocapsules to water or to an aqueous medium having pH above 5.0, the slow dissolution of the polymer, while the general arrangement of the insoluble polymer is essentially retained. In other words, the slow dissolution of the “non-insoluble” polymer results in the formation of channel-like pathways in a polymer “skeleton” formed from the water insoluble polymer, through which the active agent may escape the nanocapsule. In order to facilitate the controlled release of the active agent from the nanocapsules, it has been envisaged that one ratio between the first polymer, i.e. water insoluble polymer, and the so-called “non-insoluble” polymer is that in favor of the polymer soluble at a pH above about 5.0 (e.g. a weight:weight ratio of 75:25 in favor of the water non-insoluble polymer).

According to some embodiments, the polymeric combination comprises a mixture of a first polymer or group of polymers (the insoluble polymer) selected from Eudragit RL or Eudragit RS or a combination of same, and a second polymer or group of polymers (the water soluble or polymer soluble at a pH above 5.0) selected from Eudragit L100 55 and hydroxypropyl methylcellulose phthalate (HPMPC) or a combination of same. A specific selection of polymers combination in accordance with the invention comprises Eudragit RS and Eudragit L100-55 at a weight/weight ratio of from about 25:75 to about 50:50.

In some embodiments, the active agent is a non-hydrophilic active agent. In accordance with some embodiments, the active agent is any medicinal, cosmetic or diagnostic substance that, following oral administration, its blood bioavailability is decreased or inhibited as a result the P-gp efflux mechanism. P-gp substrates may be categorized according to their solubility and level of metabolism. A non-limiting list of P-gp substrates according to this classification includes:

High solubility and extensive metabolism: amitryptyline, cochicine, dexamethasone, diltiazem, ethinyl estradiol;

Low solubility and extensive metabolism: atorvastatin, azithromycin, carbamazepine, cyclosporine, glyburide, haloperidol, itraconazole, tacrolimus sirolimus, ritonavir, sanquinavir, lovastatin.

High solubility and poor metabolism: amiloide, amoxicillin, chloroquine, ciprofloxacin, dicloxacillin, erythromycin, fexofenadine, levodopa, midazolam, morphine, nifedipine, primaquine, promazine, promethazine, quinidine, quinine; and

Low solubility and poor metabolism—ciprofloxacin and talinolol.

The active agent may be in free acid, free base or salt form, and mixtures of active agents may be used.

In accordance with some embodiment, the active agent is a lipophilic agent. The term “lipophilic agent” is used herein to denote any compound that has a log P (octanol/water)>2.0-3.0 and a triglyceride (TG) solubility, as measured, for example, by solubility in soybean oil or similar, in excess of 10 mg/mL. This definition includes medium lipophilic drugs i.e. having a log P between 3.0 to 6, as well as highly lipophilic drugs, having a log P greater than 6.

Examples of medium to lipophilic therapeutically active agents which can be suitable for entrapment in the microcapsules according to the present disclosure include the following:

Analgesics and anti-inflammatory agents: aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac.

Anthelmintics: albendazole, bephenium hydroxynaphthoate, cambendazole, dichlorophen, ivermectin, mebendazole, oxamniquine, oxfendazole, oxantel embonate, praziquantel, pyrantel embonate, thiabendazole.

Anti-arrhythmic agents: amiodarone, disopyramide, flecainide acetate, quinidine sulphate.

Anti-bacterial agents: benethamine penicillin, cinoxacin, ciprofloxacin, clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim.

Anti-coagulants: dicoumarol, dipyridamole, nicoumalone, phenindione.

Anti-depressants: amoxapine, maprotiline, mianserin, nortriptyline, trazodone, trimipramine maleate.

Anti-diabetics: acetohexamide, chlorpropamide, glibenclamide, gliclazide, glipizide, tolazamide, tolbutamide.

Anti-epileptics: beclamide, carbamazepine, clonazepam, ethotoin, methoin, methsuximide, methylphenobarbitone, oxcarbazepine, paramethadione, phenacemide, phenobarbitone, phenyloin, phensuximide, primidone, sulthiame, valproic acid.

Anti-fungal agents: amphotericin, butoconazole nitrate, clotrimazole, econazole nitrate, fluconazole, flucytosine, griseofulvin, itraconazole, ketoconazole, miconazole, natamycin, nystatin, sulconazole nitrate, terbinafine, terconazole, tioconazole, undecenoic acid.

Anti-gout agents: allopurinol, probenecid, sulphin-pyrazone.

Anti-hypertensive agents: amlodipine, benidipine, darodipine, dilitazem, diazoxide, felodipine, guanabenz acetate, isradipine, minoxidil, nicardipine, nifedipine, nimodipine, phenoxybenzamine, prazosin, reserpine, terazosin.

Anti-malarials: amodiaquine, chloroquine, chlorproguanil, halofantrine, mefloquine, proguanil, pyrimethamine, quinine sulphate.

Anti-migraine agents: dihydroergotamine mesylate, ergotamine tartrate, methysergide maleate, pizotifen maleate, sumatriptan succinate.

Anti-muscarinic agents: atropine, benzhexol, biperiden, ethopropazine, hyoscyamine, mepenzolate bromide, oxyphencylcimine, tropicamide.

Anti-neoplastic agents and Immunosuppressants: aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine, tamoxifen citrate, testolactone.

Tacrolimus and sirolimus.

Anti-protazoal agents: benznidazole, clioquinol, decoquinate, diiodohydroxyquinoline, diloxanide furoate, dinitolmide, furzoli done, metronidazole, nimorazole, nitrofurazone, ornidazole, tinidazole.

Anti-thyroid agents: carbimazole, propylthiouracil.

Alixiolytic, sedatives, hypnotics and neuroleptics: alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, baloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone.

beta-Blockers: acebutolol, alprenolol, atenolol, labetalol, metoprolol, nadolol, oxprenolol, pindolol, propranolol.

Cardiac Inotropic agents: amrinone, digitoxin, digoxin, enoximone, lanatoside C, medigoxin.

Corticosteroids: beclomethasone, betamethasone, budesonide, cortisone acetate, desoxymethasone, dexamethasone, fludrocortisone acetate, flunisolide, flucortolone, fluticasone propionate, hydrocortisone, methylprednisolone, prednisolone, prednisone, triamcinolone.

Diuretics: acetazolamide, amiloride, bendrofluazide, bumetanide, chlorothiazide, chlorthalidone, ethacrynic acid, frusemide, metolazone, spironolactone, triamterene.

Anti-parkinsonian agents: bromocriptine mesylate, lysuride maleate.

Gastro-intestinal agents: bisacodyl, cimetidine, cisapride, diphenoxylate, domperidone, famotidine, loperamide, mesalazine, nizatidine, omeprazole, ondansetron, ranitidine, sulphasalazine.

Histamine H,-Receptor Antagonists: acrivastine, astemizole, cinnarizine, cyclizine, cyproheptadine, dimenhydrinate, flunarizine, loratadine, meclozine, oxatomide, terfenadine.

Lipid regulating agents: bezafibrate, clofibrate, fenofibrate, gemfibrozil, probucol.

Nitrates and other anti-anginal agents: amyl nitrate, glyceryl trinitrate, isosorbide dinitrate, isosorbide mononitrate, pentaerythritol tetranitrate.

Nutritional agents: betacarotene, vitamin A, vitamin B.sub.2, vitamin D, vitamin E, vitamin K.

HIV protease inhibitors: Nelfinavir,

Opioid analgesics: codeine, dextropropyoxyphene, diamorphine, dihydrocodeine, meptazinol, methadone, morphine, nalbuphine, pentazocine.

Sex hormones: clomiphene citrate, danazol, ethinyl estradiol, medroxyprogesterone acetate, mestranol, methyltestosterone, norethisterone, norgestrel, estradiol, conjugated oestrogens, progesterone, stanozolol, stibestrol, testosterone, tibolone.

Stimulants: amphetamine, dexamphetamine, dexfenfluramine, fenfluramine, mazindol.

Without being limited thereto, active agents in accordance with the present disclosure include tacrolimus, sirolimus halofantrine, ritonavir, lopinavir, amprenavir, saquinavir, calcitrol, dronabinol, isotretinoin, tretinoin, risperidone base, valproic acid while pro-drugs include dexamethasone palmitate, paclitaxel palmitate, docetaxel palmitate.

Some non-limiting examples of lipophilic drugs which can be incorporated into the composition disclosed herein and their medical applications are described by Robert G. Strickley [Strickley R. G. Pharmaceutical Research, 21(2):201-230; (2004)] and by Kopparam Manjunath, et al. [Manjunath K. et al., Journal of Controlled Release 107:215-228; (2005)].

In accordance with another embodiment, the active agent is an amphipathic agent. The term “amphipathic agent” is used herein to denote any compound that has a log P value between 1.5-2.5 and a triglyceride (TG) solubility, as measured, for example, by solubility in soybean oil or similar, in excess of 10 mg/mL.

Examples of amphipathic active agents which may be delivered by the composition disclosed herein include, without being limited thereto, pysostigmine salicylate, chlorpromazine, fluphenazine, trifluoperazine, and lidocaine, bupivacaine, amphotericin B, etoposide, teniposide and antifungal echinocandins and azoles, such as clotrimazole and itaconazole.

Another example of a therapeutically, non-hydrophilic active agent suitable for entrapment in the microcapsules disclosed herein includes, without being limited thereto, is clozapine. Clozapine is an effective atypical antipsychotic drug applied in the treatment of resistant schizophrenia. Clozapine is rapidly absorbed orally with a bioavailability of 27%. Clozapine is extensively metabolized by hepatic microsomal enzymes (CYP1A2 and CYP3A4) and forms N-demethyl and N-oxide metabolites.

Another example of an active agent suitable for entrapment in the microcapsules disclosed herein includes, without being limited thereto include any active agent that is soluble in the oil core. Methods to determine the solubility of the active agent in the oil core, and the composition of the oil core, are readily determined by one of ordinary skill in the art. A person of ordinary skill in the art would recognize that even hydrophilic active agents can be incorporated into the oil core, provided the hydrophiling agent is either soluble in an oil, or capable of being modified to be soluble in an oil.

As a person versed in the art would understand, the “hydrophilicity” of the materials is a characteristic of materials exhibiting affinity for water, while the “hydrophobic” materials possess the opposite response to water.

In some embodiments, the material hydrophobicity or hydrophilicity may be due to the material's intrinsic behaviors towards water, or may be achieved (or tuned) by modifying the material by one or more of cross-linking the material, derivatization of the material, charge induction to the material (rendering it positively or negatively charged), complexing or conjugating the material to another material and by any other means known in the art.

Thus, in accordance with the present invention, the selection of a material may be based on the material intrinsic properties or based on the material's ability to undergo such aforementioned modification to render it more or less hydrophobic or hydrophilic. In some embodiments, the nanoparticle material and/or the nanocarrier material may be cross-linked in order to reduce material hydrophilicity (decrease solubility in aqueous media).

In some embodiments, the active agent that is soluble in the oil core is a peptide. In some embodiments, the solubility of the peptide in a selection of oils is determined, and the oils in which the peptide exhibits the greatest solubility is selected to dissolve the peptide and form the spherical microcapsules according to some embodiments of the present invention.

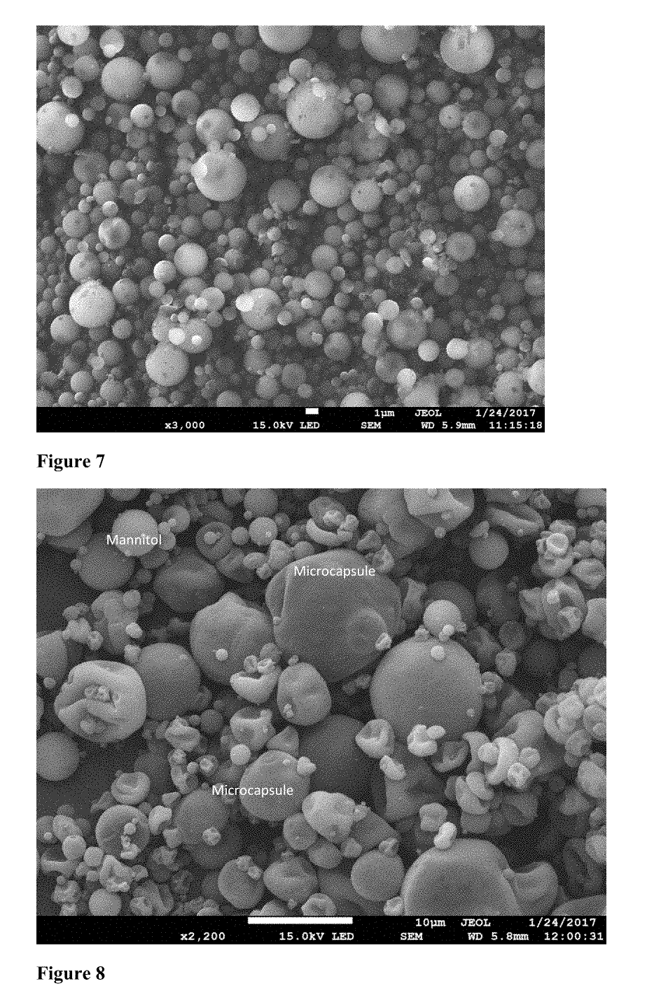

FIG. 8 is a high resolution SEM image ×2200 of a nanocapsule in microcapsule formulation of the hydrophilic polypeptide, exenatide. Exenatide is a polypeptide with molecular weight of 4186 Daltons which is significantly higher than the hydrophilic peptide (panel E) which is 11924 Daltons. Therefore the formulation of exenatide was based on the suspension (not solubilization) of the polypeptide in the nanoencapsulation procedure. As such; residual free exenatide (nonencapsulated in the nanocapsules) remains in the microcapsules and therefore may affect the microcapsules morphology.

In some embodiments, the oil core comprises the peptide or a salt thereof, and one or more oil. In some embodiments, the oil core is such that the peptide (or salt thereof) is solubilized within the one or more oil, such that a homogenous formulation is obtained.

In order to facilitate formation of nanocapsules of the invention, the oil formulation may, in some embodiments, further comprise at least one surfactant.

The term surfactant should be understood to encompass any agent that is capable of lowering the surface tension of a liquid, allowing for the formation of a homogeneous mixture of at least one type of liquid with at least one other type of liquid, or between at least one liquid and at least one solid. Thus, surfactants used in the oil core of the invention may be used to control the surface tension and surface interaction between the oil core and its surroundings, thereby assisting in the process of forming the nanocapsules.

Non-limiting examples of suitable surfactants are oleoyl polyoxyl-6glycerides NF (Labrafil M1944 CS, Gatefosse) and all nonionic oil surfactants exhibiting an HLP from 8 to 10.

In some embodiments, an organic phase is first obtained by mixing the peptide-containing oil core with a solution of the hydrophobic polymer in a solvent. The solvent, in some embodiments, may be acetone.

### Bioavailability

In some embodiments, the present invention provides a method of increasing bioavailability of an active agent in a human subject's body, the method comprising administering to the subject spherical microcapsules, as described in this disclosure, comprising a plurality of nanocapsules situated within a lumen formed by the shell of the spherical microcapsule, the nanocapsules comprising an oil core carrying an active agent and a polymeric shell coating.

In some embodiments, the spherical microcapsules are particularly useful for gastrointestinal delivery of active agents. In some embodiments, the active agent is a substrate of P-gp efflux pump. The term “substrate of the P-gp efflux pump” which may be used interchangeably with the term “P-gp substrate” as used herein refers to any active substance (for therapeutic, cosmetic or diagnostic purposes) that is subject to active transport, “efflux” out of cells via the P-gp membrane bound transporter. The P-gp is expressed along the entire length of the gut and also in the liver, kidney, blood brain barrier and placenta. In this context, the present disclosure concerns medicinal substances subjected to active transport by the intestinal P-gp which is located on the apical membranes of the epithelial cells. Utilizing the energy that is generated by hydrolysis of ATP, P-gp drives the efflux of various substrates against a concentration gradient and thus reduce their intracellular concentration and in the case of active substances, their oral bioavailability.

Without being limited by theory, it has been found that the resulting oral bioavailability of the encapsulated active agent from the spherical microcapsules was significantly enhanced above the encapsulated active agent in irregular microcapsules (not symmetrically spherical) of the same composition (same type and amount of active agent and polymers forming the microcapsules in both formulations). The difference in morphology was evident from scanning electron microscope at various magnifications, including ×20000.

In some embodiments, the improvement in bioavailability is exhibited by enhanced absorption of the encapsulated active agent from the gastrointestinal tract to the blood.

In some embodiments, the present invention provides a method of treating a subject for a pathological condition. The method comprises administering to the subject spherical microcapsules, as described in this disclosure, comprising a plurality of nanocapsules situated within a lumen formed by the shell of the spherical microcapsule, the nanocapsules comprising an oil core carrying an active agent and a polymeric shell coating wherein the administration provides an amount of active agent sufficient to treat the pathological condition.

The spherical microcapsules, according to some embodiments, may be formulated in accordance with any desired application. There are almost limitless applications for such spherical microencapsulated material. Depending inter alia, on the active agent, the composition may be applicable in agriculture, pharmaceuticals, foods, cosmetics and fragrances, textiles, paper, paints, coatings and adhesives, printing applications, and many other industries.

In accordance with some embodiments, the composition is for use in medicine, cosmetics or diagnosis.

In yet some further embodiments, the composition is formulated as a pharmaceutical dosage form, the dosage form comprising, in addition to the composition a physiologically acceptable carrier.

In some embodiments, the physiologically acceptable carrier is suitable for oral administration. To this end, the dry composition may be included in an enteric vehicle, such as an enteric capsule. Non-limiting examples of enteric capsules include soft or hard entero-coated capsules as known in the art.

For oral delivery tablets, pills, powders, lozenges, sachets, cachets, elixirs, suspensions, aerosols (as a solid or in a liquid medium), and sterile packaged powders as well as other delivery forms can be used.

Compositions comprising spherical microcapsules according to some embodiments of the present invention were shown to provide elevated blood levels of the active agents exemplified as compared to microcapsules having irregular shape, i.e. produced by the same step, albeit without a prior spray drying the bulk agent (e.g. sugar) in the evaporator.

In yet some embodiments, the pharmaceutical dosage form is formulated for administration by injection.

According to some embodiments, the pharmaceutical dosage form disclosed herein provides controlled release of the active agent. As used herein, “controlled release” means any type of release which is not immediate release. For example, controlled release can be designed as modified, extended, sustained, delayed, prolonged, or constant (i.e. zero-order) release. In theory, one of the most useful release profiles is constant release over a predetermined period of time.

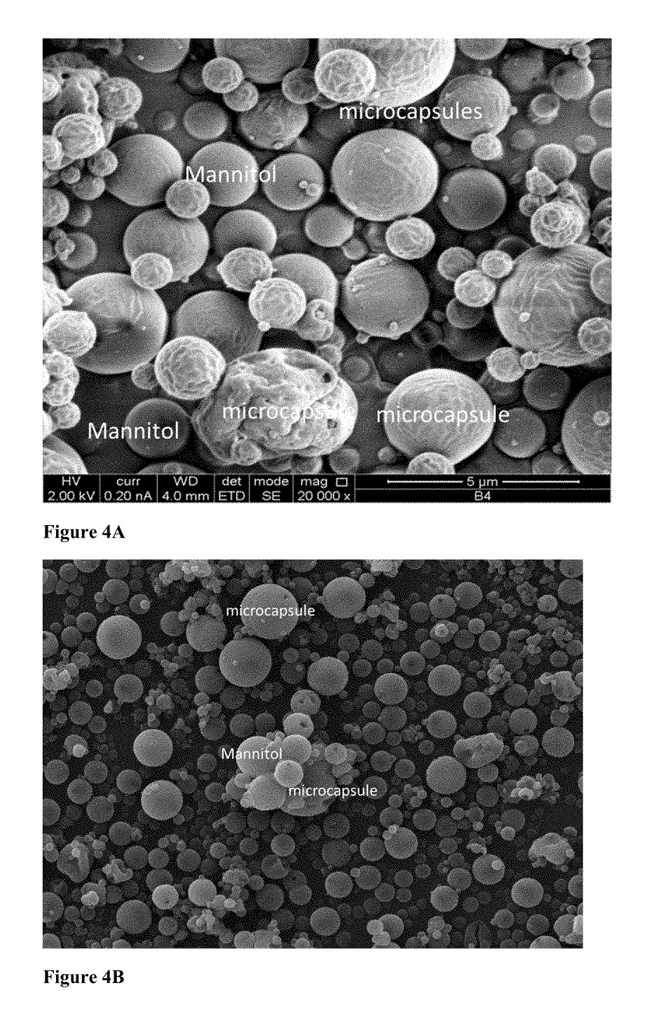

The present disclosure also provides a method of increasing bioavailability of an active agent in a human subject's body, the method comprises providing the subject with the composition disclosed herein (comprising spherical microcapsules). The results presented herein show that, by the use of the spherical microcapsule, bioavailability of the tested active agents in the blood increases, at least by a factor of 2, with respect to control irregularly shaped microcapsules (See FIG. 5).

The present disclosure also provides a method of treating a subject for a pathological condition which requires for the treatment an effective amount of an active agent within the subject's blood system, the method comprises providing the subject with the composition disclosed herein.

The term “pathological condition” used herein denotes any condition which requires for improving the well-being of the subject the delivery, of an active agent being a drug or pro-drug or diagnostic agent, such as those listed hereinabove.

In some embodiments, when the active agent is a non-hydrophilic entity, such as, without being limited thereto, a lipophilic agent, or any lipophilic derivative of an active agent, the delivery of the active agent in accordance with the present disclosure is via the intestines. In some embodiments, the delivery of the active agent is via the intestinal paracellular, trancellular or lymphatic transport, or a combination thereof. The non-limiting list of conditions includes, inter alia, inflammation and autoimmune disorders, parasitism (e.g. malaria) bacterial, viral or fungal infection, cardiac disorders (e.g. arrhythmia), coagulation disorders, depression, diabetics, epilepsy, migraine, cancer, immune disorders, hormonal disorders, psychiatric conditions, gastrointestinal tract disorders, nutritional disorders, and many others, as known in the art.

In some embodiments, when the active agent is a hydrophilic active agent dissolved or suspended in the oil core, the delivery of the active agent in accordance with the present disclosure is via the intestines. In some embodiments, the delivery of the active agent is via the intestinal paracellular, trancellular or lymphatic transport, or a combination thereof. The non-limiting list of conditions includes, inter alia, inflammation and autoimmune disorders, parasitism (e.g. malaria) bacterial, viral or fungal infection, cardiac disorders (e.g. arrhythmia), coagulation disorders, depression, diabetics, epilepsy, migraine, cancer, immune disorders, hormonal disorders, psychiatric conditions, gastrointestinal tract disorders, nutritional disorders, and many others, as known in the art.

The effective amount of active agent in the pharmaceutical dosage form can vary or be adjusted widely depending upon the particular application, the manner or introduction, the potency of the particular active agent, the loading (or not) of the agent into nanocapsules, and the desired concentration. The effective amount is typically determined in appropriately designed clinical trials (dose range studies) and the person versed in the art will know how to properly conduct such trials in order to determine the effective amount. As generally known, an effective amount depends on a variety of factors including the hydrophobicity of the active agent and when relevant, the lipophilicity, the selection of polymers forming the nanocapsule (the oil droplet's coating) and/or the outer gel forming envelop, the distribution profile of the active agent within the body, a variety of pharmacological parameters such as half-life in the body, on undesired side effects, if any, on factors such as age and gender, etc.

The pharmaceutical dosage form can be administered over an extended period of time in a single daily dose, in several doses a day, as a single dose and in several days, etc. The treatment period will generally have a length proportional to the length of the disease process and the specific microsphere's effectiveness (e.g. effective delivery via the intestines, effectiveness of the agent etc.) and the patient species being treated.

The present invention is further illustrated, but not limited by, the following examples.

### EXAMPLES

As will be shown in the following non-limiting examples, the effect of the spherical morphology (vs. the irregular morphology obtained without the addition of the bulk agent, e.g. D-mannitol) was evaluated. More specifically, the pharmacokinetics of the active agent accommodated in the microcapsules (irregular shape vs. spherical shape) following oral administration to rats was examined. The assumption, based on hitherto known art was that the spherical microcapsules will reduce the absorption of the encapsulated active agent via the gastrointestinal tract due to their reduced surface area in comparison to irregular surfaces. The concern was that with the decreased surface area of the spherical microcapsules, the interaction between the surfaces of the microcapsules with the gastrointestinal mucosa will be reduced as well. Surprisingly, it was found that the uniform and spherical microcapsules exhibited a significant increase of oral bioavailability much above (at least twice) that of the irregular microcapsules.

One of ordinary skill in the art can readily appreciate that the process disclosed below, using a small-scale evaporator can be scaled up to a larger scale, or industrial scale.

### Materials and Methods

### Materials

D-Mannitol EP, FCC, USP was purchased from Sigma Aldrich. PLGA (Lactel end capped hexandiol 50:50 intrinsic viscosity 0.15-0.25 dL/g, lot No. A11-045, expiry date May 17, 2015);

Poly(methacrylic acid), Ethyl acrylate 1:1 (Eudragit® L100-55) was obtained from Evonik Rohm (Kirschenallee, Germany);

Hydroxypropylmethylcellulose (Methocel E4M Premium) was purchased from Dow Chemical Company (Midland, Mich., USA);

Oliec acid was purchased from Fisher Scientific UK. Octanoic acid was purchased from Merck KGaA (Germany);

Sodium phosphate monobasic, monohydrate was purchased from Mallinckrodt Chemicals (Phillipsburg, N.J., USA);

Phosphate buffered saline was obtained from Biological Industries (Kibbutz Beit Haemek, Israel);

All organic solvents were HPLC grade and purchased from J.T. Baker (Deventer, Holland);

Lopinavir (99.6% purity) pharma grade API (Active Pharmaceutical Ingridient) was purchased from Aurobindo Pharma Ltd. India.

### Techniques

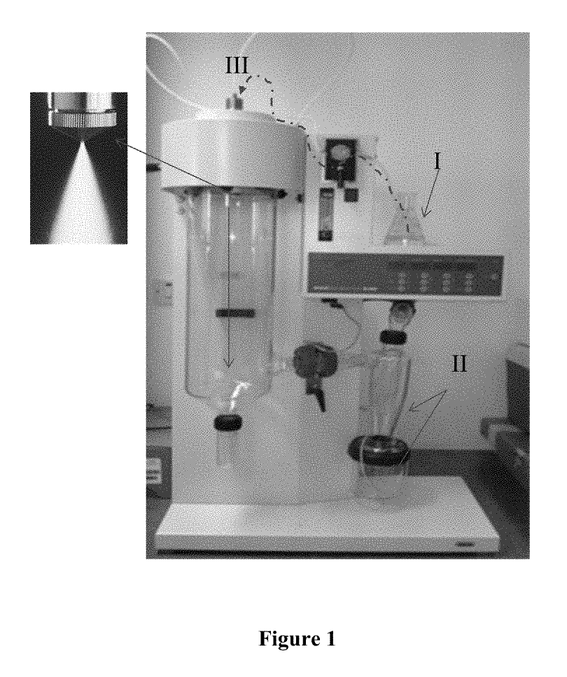

The nanocapsules within microcapsules were prepared using a spray drying technique. FIG. 1 is an image of a spray drying system used in accordance with this example (Buchi mini-sprayer dryer Model B-290), showing specifically the beaker (I) containing a suspension of nanocapsules (NC) in a polymeric solution, the tube directing the suspension into the nozzle (dashed arrow), and the cyclone (II) where the spray dried powder is collected and the nozzle (III) from which the suspension is spray dried.

### Methods

In all examples below microcapsules contained nanocapsules prepared as described below:

### Nanocapsule (NC) Preparation

The primary NCs were first prepared by dissolving 1500 mg OA (oleic acid), 300 mg labrafil M 1944 CS, 300 mg PLGA and 450 mg LPV in 300 ml of acetone. Then, 210 ml of water (Double Distilled Water—DDW) were added slowly to the oil phase, creating an o/w emulsion, as evidenced by the rapid formation of opalescence in the suspension medium.

Separately, the NCs were prepared by dissolving in 100 mL of acetone: 300 mg of PLGA (Lactel end capped hexandiol 50:50 intrinsic viscosity 0.15-0.25 dL/g). In separate glass vials, 25 mg of an eight amino acid peptide was dissolved in 120 mg of octanoic acid with vortex for 5 minutes at room temperature. To the same vial, 380 mg of oleic acid and 100 mg of Labrafil M 1944 CS were added and then vortexed vigorously for 2 minutes until complete dissolution. The contents of the vial were transferred to the acetone solution and washed three times with acetone to confirm that all the oil solution was added to the acetone-PLGA solution. 70 mL of bi-distilled water were slowly added to the organic phase while stirring at 1000 rpm until an O/W (oil-in-water) suspension formed (NCs suspension).

### Example 1: Preparation of Irregular Microcapsules

Microcapsules were formed by microencapsulating either lopinavir or an eight amino acid peptide or a polypeptide of 4168 dalton which was encapsulated in nanocapsules (NCs) using the spray-drying technique using a system as shown in FIG. 1.

Specifically, 132.26 mg of NaH₂PO₄.H₂O were dissolved in 150 ml of doubled distilled water (DDW) and pH was adjusted to 6.5 using NaOH 1N. To this solution, 750 mg of Eudragit L 100-55 were added and pH was adjusted again in the same manner to 6.5. Then 100 ml of HPMC solution were prepared by first dispersing 1 g of HPMC in 100 ml of DDW at about 80° C., and then cooled to room temperature under stirring to dissolution. The Eudragit L 100-55 at pH of 6.5 was added to HPMC solution and the combined mixture of Eudragit+HPMC was poured via funnel to the NCs dispersion medium at room temperature. The combined mixture of NCs with the polymer solution of HPMC+Eudragit was transfer to rotor evaporator and the acetone was evaporated under room temperature. At the end of acetone evaporation an equal volume (by weight) of DDW was added to obtain total volume of 760 g.

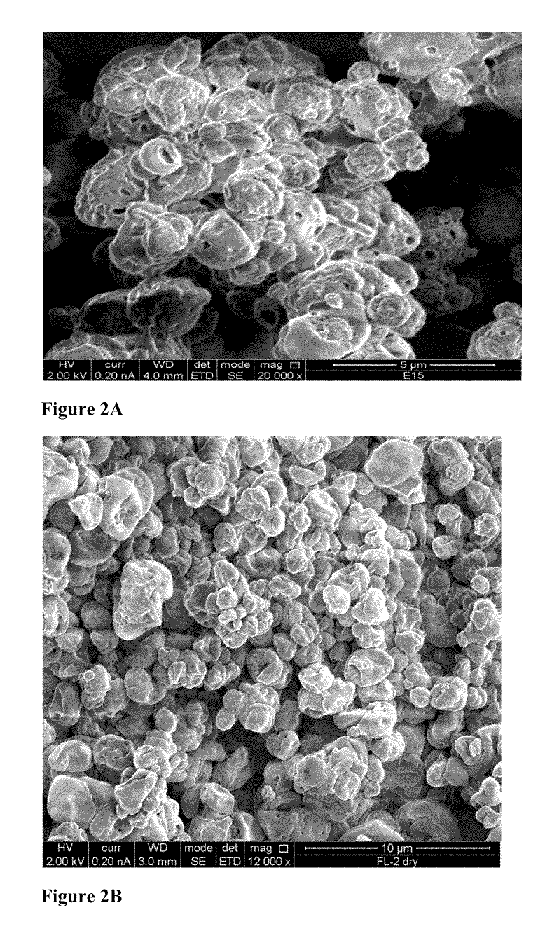

The morphology of the reference formulations is depicted in FIGS. 2A-2E, evidencing their irregular morphology. Microcapsules containing lopinavir are shown in FIGS. 2A-D. Microcapsules containing an eight amino acid peptide are shown in FIG. 2E.

### Example 2: Preparation of Spherical Microcapsules

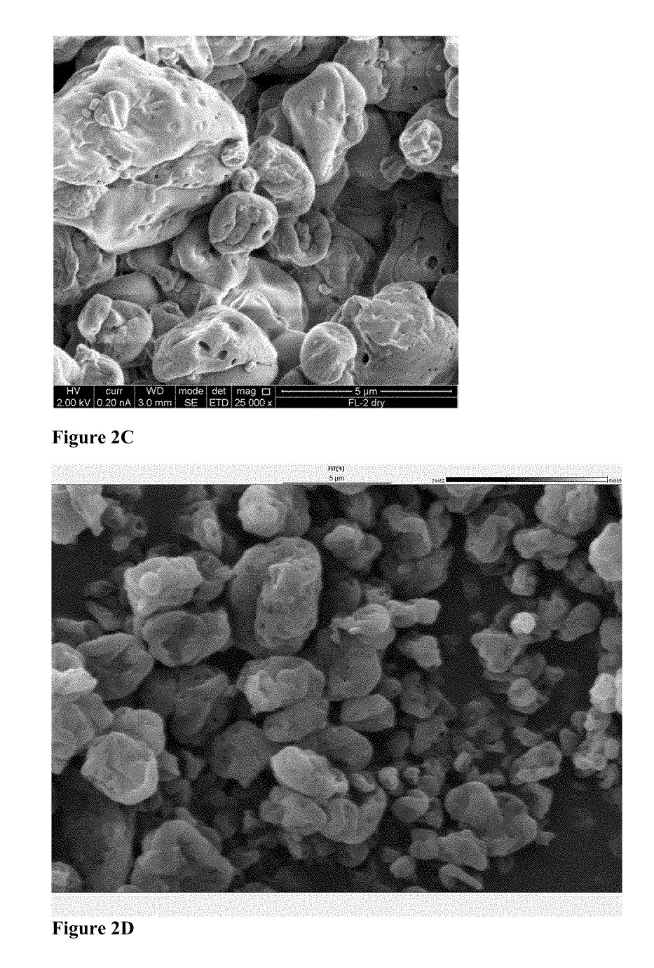

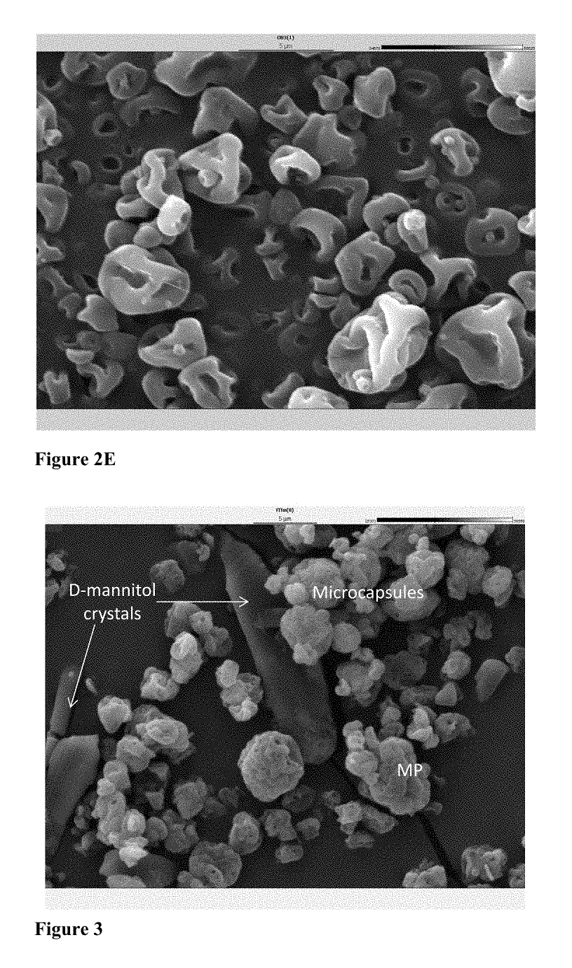

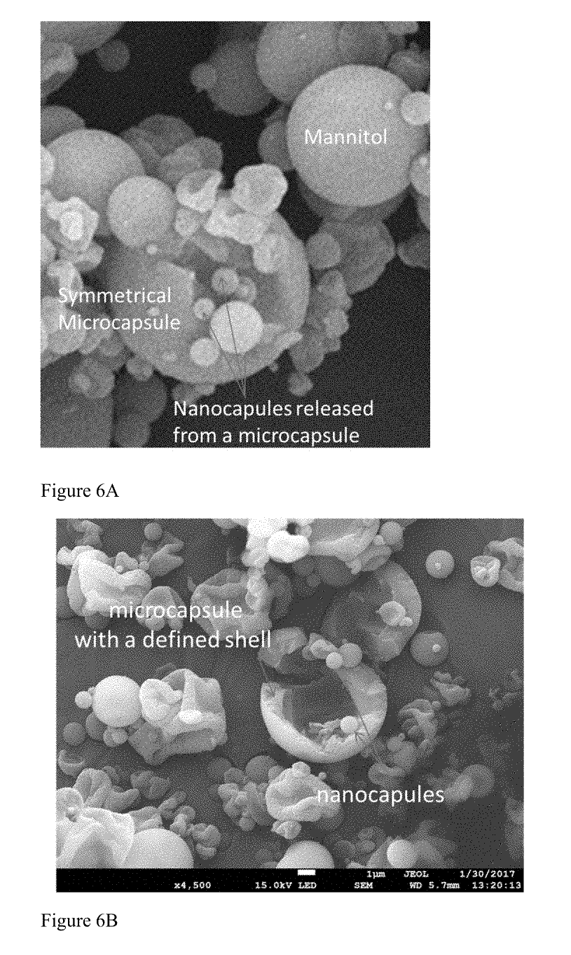

The spherical microcapsules according to the present disclosure were prepared in combination with D-mannitol. Specifically, Spray drying of D-mannitol was performed prior to the spray drying of the material for forming the NCs/microcapsules polymers suspension as described in Example 1. FIG. 7 is an SEM image of mannitol particles used as a control to distinguish between the morphology and size of mannitol particles in comparison to the microcapsules as depicted in FIGS. 4A, 4B and 4C. The rationale was to reduce the inherent surface energy of the interior surfaces of the cyclone and the receiver flask and by that to prevent an immediate adsorption of the dry microcapsules to the interior collection surfaces. Without being bound by theory, it was assumed that the irregular morphology of the microcapsules (without mannitol) is mainly due to their adsorption to the interior collection surfaces during their spray drying. Therefore, a D-mannitol solution of 1% w/v in DDW (100 ml) was spray dried by a Buchi mini spray-drier B-290 apparatus (Flawil, Switzerland) under the following conditions: Inlet temperature 110° C.; outlet temperature 50° C.; aspiration 100%; pump rate 20% and nozzle cleaner 2. The dried D-mannitol powder was accumulated/adsorbed on the surface of the cyclone separator and the receiver flask surfaces. The morphology of the innovative formulation is depicted in FIG. 3.

### Example 3: Preparation of Mannitol Fine Powder—Microcapsule Combination (Control Formulation)

In order to show that the effect of mannitol on the morphology of microcapsules is specific to spray drying procedure and has no effect on the delivery of the drug per se, a fine powder of mannitol (grinned by pastel and mortar) was added and mixed with a sample of the irregular MC (prepared according to Example 1). The irregular MC was premixed for 3 hours (h) by continuous slow magnetic stirring at 200 rpm. The morphology of this mixed sample is shown in FIGS. 4A-4C.

### Example 4: Spherical Microcapsules

Encapsulation of NCs in microcapsules was obtained by the spray drying of the NCs in HPMC/Eudragit polymers blend as described in Example 1. However, the suspension was spray-dried immediately after the spray drying of mannitol under the following conditions: Inlet temperature 160° C.; outlet temperature 98° C.; aspiration 100%, pump rate 20% (the feeding rate was 4 ml/min) and nozzle cleaner 2. The powder of the microcapsules with D-mannitol was accumulated in the cyclone separator and collected later. The average yield of the process was 50-70%.

### Physicochemical Characterization of the Spherical Microcapsules

### Drug Content in the Final Dried Microcapsule Formulations

Samples of 10 mg were taken from the formulation. The samples were completely dissolved in 3 ml DMSO, in a volumetric flask under agitation for 1 h and the volume was then adjusted to 10 ml with methanol. 975 μl were withdrawn from each flask and the volume was completed to 1000 μl with internal standard (10 μg) solution of diazepam in methanol. Finally, for all formulations, the drug content was determined by injecting 20 μl from each sample into an HPLC device, under the following conditions: Mobile phase—ACN:H2O 45:55, flow rate—0.8 ml/min, wavelength—210 nm, column—XTerra MS C8 5 μm 3.9×150 mm purchased from Waters (Milford, Mass., USA, distributed by Medtechnica. Petach-Tikva, Israel). Two calibration curves were constructed from LPV concentrations ranging between 0 to 200 μg/ml and internal standard diazepam at a concentration of 10 μg/ml. The samples of the first calibration curve were dissolved in methanol and the samples of the second curve were dissolved in DMSO:MeOH 3:7 as were the formulation samples. The calculated recovery percentage was 100%.

### Mannitol Content of the Final Dried Microcapsule Formulations

Mannitol content is assayed by commercial colorimetric assay kits as an example D-mannitol colorimetric assay kit—MAK096 Sigma Aldrich. Briefly: D-Mannitol Colorimetric Assay kit provides a simple and direct procedure for measuring D-mannitol in a variety of samples, including fruit and juices. D-Mannitol concentration is determined by a coupled enzyme assay, which results in a colorimetric (450 nm) product proportional to the D-mannitol present. The sugar alcohol L-arabitol also acts as a substrate for this assay. Therefore, this kit can be used to measure L-arabitol.

### Morphological Evaluation—SEM

Morphological evaluation of spray-dried NC-loaded microspheres was carried out using High-Resolution Scanning Electron Microscope (Sirion, HR-SEM; FEI Company, The Netherlands). The specimens were fixed on an SEM-stub using double-sided adhesive carbon tape or alternatively.

The SEM micrograph (magnification ×2000) of microcapsules bearing a drug (Lopinavir) loaded NCs as a result from their spray-drying after spray drying of D-mannitol (1% w/v in water) is provided in FIG. 3. As observed the microcapsules plus mannitol have a well-defined symmetrical spherical morphology.

In comparison, irregular microcapsules were prepared without the D-mannitol (in accordance with the procedure of U.S. Pat. No. 9,023,386). Typical SEM micrographs of such Reference microcapsules are shown in FIGS. 2A-2E (magnifications ×20000, ×12000, ×25000, ×50000, and ×3500, respectively). FIGS. 2A-2E depict the irregular morphology of the spray-dried microcapsules of two independent batches at various magnifications.

In addition, FIG. 4A-4C shows SEM micrograph of the irregular microcapsules with D-mannitol added after their production as fine powder to the microcapsule powder. The D-mannitol crystals are observed near the irregular microcapsules. Hence, the mixture of D-mannitol fine powder with the solid spray dried microcapsules didn't modify the morphology of the spray dried irregular microcapsules.

### In Vivo Studies—Pharmacokinetic Studies in Rats

All the animal studies in this research were carried out in accordance with the rules and guidelines concerning the care and use of laboratory animals and were approved by the Israeli ethical committee.

Sprague Dawley male rats weighing 300-320 g were used in this study and were separated randomly into to evaluate the biofate of LPV in different formulations. The animals were housed in SPF conditions, fasted overnight (12-14 h prior to the experiment) with free access to water. The animals were given a dose of 10 mg/kg LPV. All the microparticulate formulations, I prepared by dispersing the NCs embedded in microparticles in DDW to the final concentration of 32 mg/ml. Blood samples (400-500 μl) were withdrawn from the tail vein at 0, 0.5, 1, 2, 4, 8, 12 and 24 h. Saline solution was given to the animals after 30 min and then again after 3 h. The blood samples were collected in heparin containing tubes. The samples were immediately centrifuged at 10,000 rpm for 5 min, after which 200-300 μl of plasma samples were transferred to new tubes and stored at −80° C. until further analysis by LC-MS/MS as described below.

The blood samples were treated by protein precipitation in methanol, following 15 min centrifuge at 10,000 rpm. 25 ng of quinoxaline (QX) dissolved in methanol were added to each sample, as an internal standard. The supernatant layer was collected and the samples were injected into an LC-MS/MS device under the following conditions: A Phenomenex Kenetex, column (RP-C18, 50×2.1 mm, 2.6 μm, 100A) in gradient mode. The mobile phase consisted of A=methanol/formic acid 99.9/0.1 and B=water/formic acid 99.9/0.1. The A/B ratio is 58/42 at t=0 min, during the first 1.5 min the ratio changed gradually to A/B 80/20 and remained steady until 2.1 min. The ratio changed again to A/B 58/42 rapidly from 2.1-2.15 min and remained constant until the end of the run at 5 min, for the purpose of system cleaning and stabilization. The flow rate was maintained at 0.35 ml/min, and the column temperature was maintained at 35° C. LC-MS/MS analysis was performed with a thermo scientific Accela HPLC system coupled with a TSQ Quantum Access MAX detector in positive ionization mode. Detection and quantification were carried out by multiple-reaction monitoring with transitions from m/z 629.3 to 156 for LPV and from m/z 313.1 to 246 for QX. The tested samples were quantified against a calibration curve in the range of 0-100 ng/ml. The correlation coefficient values were higher than 0.99 indicating that good linearity, accuracy and specificity were achieved.

The comparative pharmacokinetics profiles of the spray dried microcapsules with and without D-mannitol (irregular and spherical morphology respectively) is provided in FIG. 5. Specifically, the microcapsule powder was suspended in water and administered to rats by gavage by equal dose of 10 mg/kg of the encapsulated drug lopinavir. Two batches of microcapsules without D-mannitol (B1 and B3) were compared to two batches of microcapsules with spray dried D mannitol (B4 and B7), the formulation of the NCs and the spray drying conditions (inlet and outlet temperatures and air flow) were the same for all batches. The only variable is the spray drying of D-mannitol before the spray drying of the microcapsules (in batches B4 and B7). The formulations B1 and B3 were produced without the pre-spray-drying of Mannitol, whereas B4 and B7 included the pre-spray-drying of mannitol. FIG. 5 shows that plasma levels of the MC with D-mannitol is much higher as compared to the blood level of the irregular MC (prepared without prior spraying with D-mannitol).

The comparative AUC (area under the plasma lopinavir time curve) values of the tested groups as for extent of absorption is provided in Table 1 below.

TABLE 1
AUC

| Batch | AUC |
|---|---|
| B7 (n = 5) | 2452 |
| B4 (n = 3) | 2104 |
| B1 (n = 3) | 1247 |
| B3 (n = 3) | 883 |

The microcapsules with the prior spray drying of D-mannitol doubled the extent of absorption above the microcapsules with the irregular morphology (without D-mannitol). The increase of Cmax was significantly higher in B4 and B7 in comparison to B1 and 3 as revealed by the paired t-test, p<0.05 as shown in Table 2 below.

TABLE 2
P values (t-test)

| Time | P values | | | |
|---|---|---|---|---|
| point (h) | B7/B1 | P values B7/B3 | P values B4/B1 | P values B4/B3 |
| 1 | 0.012067 | 0.003691 | 0.276124 | 0.009849 |
| 2 | 0.487524 | 0.215681 | 0.032535 | 0.008087 |
| 4 | 0.267686 | 0.159417 | 0.038736 | 0.079601 |
| 6 | 0.2363 | 0.092573 | 0.238343 | 0.134816 |

1. A composition comprising:
a plurality of microcapsules, each comprising a shell and a core carrying an active agent selected from the group consisting of: (a) a non-hydrophilic active agent and (b) a hydrophilic active agent dissolved or suspended in an oil;
wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

2. The composition of claim 1, wherein the non-hydrophilic active agent is a lipophilic agent.

3. The composition of claim 1, wherein the active agent is a hydrophilic agent with a molecular weight of about 1 kiloDalton or about 4 kiloDalton.

4. The composition of claim 1, further comprising a plurality of nanocapsules situated within a lumen formed by each shell of the plurality of microcapsules.

5. The composition of claim 4, wherein the plurality of nanocapsules are accommodated in a gel forming polymer.

6. The composition of claim 5, wherein the plurality of nanocapsules each comprise a shell comprising a polymeric coating and a liquid oil core comprising an active agent dissolved or suspended in the liquid oil core.

7. A method of preparing a spherical microcapsules comprising a plurality of nanocapsules, the method comprising:
(a) spray drying sugar in a spray drying evaporator comprising a spray chamber and a cyclone, the amount of sugar being sufficiently in excess to cause at least a portion of the sugar to adhere to the surfaces of the evaporator;
(b) spray drying a dispersion comprising an active agent encapsulated in oil core droplets of the plurality of nanocapsules and a gel forming polymer or a combination of gel forming polymers to obtain microcapsules, wherein the active agent is selected from the group consisting of: (a) a non-hydrophilic active agent and (b) a hydrophilic active agent dissolved or suspended in an oil;
wherein a shell of the oil core droplets comprises a polymeric coating, and wherein at least a portion of the microcapsules are spherical when viewed in a scanning electron microscope with a magnification in the range of between ×2000 and ×50000.

8. A method of preparing spherical microcapsules comprising a plurality of nanocapsules situated within a lumen formed by a shell of the spherical microcapsule, the nanocapsules comprising an oil core carrying an active agent and a polymeric shell coating, the method comprising:
(a) spray drying sugar in a spray drying evaporator, the amount of sugar being sufficiently in excess to cause at least a portion of the sugar to adhere to the interior collection surfaces of the evaporator;
(b) providing an organic phase comprising oil, a water miscible organic solvent, an active agent dissolved in the solvent and a polymer or combination of polymers for coating the oil core, wherein the active agent is selected from the group consisting of: (a) a non-hydrophilic active agent and (b) a hydrophilic active agent dissolved or suspended in an oil;
(c) slowly adding water to the organic phase to obtain an emulsion;
(d) continuously adding water to the emulsion to induce phase inversion of the emulsion thereby obtaining an oil in water (o/w) emulsion;
(e) mixing the o/w emulsion with a gel forming polymer or a combination of gel forming polymers; and
(f) removing the organic solvent by means of evaporation and water by means of spray drying to obtain the spherical microcapsules.

9. A pharmaceutical dosage form comprising:
a composition and a physiologically acceptable carrier, wherein the composition comprises a plurality of microcapsules, each comprising a shell and a core carrying an active agent selected from the group consisting of: (a) a non-hydrophilic active agent and (b) a hydrophilic active agent dissolved or suspended in an oil;
wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

10. The pharmaceutical dosage form of claim 9, further comprising a plurality of nanocapsules situated within a lumen formed by each shell of the plurality of microcapsules.

11. The pharmaceutical dosage form of claim 10, wherein the plurality of nanocapsules each comprising comprise a shell of comprising a polymeric coating and a liquid oil core comprising an active agent active agent dissolved or suspended in the liquid oil core.

12. The pharmaceutical dosage form of claim 9, wherein the physiologically acceptable carrier is suitable for oral administration or suitable for administration by injection.

13. A method of increasing bioavailability of an active agent in a human subject's body; the method comprising:
administering to the human subject a composition or a pharmaceutical dosage form comprising the composition and a physiologically acceptable carrier, wherein the composition comprises a plurality of microcapsules, each comprising a shell and a core carrying an active agent selected from the group consisting of: (a) a non-hydrophilic active agent and (b) a hydrophilic active agent dissolved or suspended in an oil;
wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

14. The method of claim 13, wherein the delivery of the active agent is via the intestinal paracellular, trancellular or lymphatic transport, or a combination thereof.

15. The method of claim 13, wherein the physiologically acceptable carrier is suitable for oral administration or suitable for administration by injection.

16. A method of treating a subject for a pathological condition which requires for the treatment an effective blood level of an active agent, the method comprising:
administering to the subject a composition or a pharmaceutical dosage form comprising the composition and a physiologically acceptable carrier, wherein the composition comprises a plurality of microcapsules, each comprising a shell and a core carrying an active agent selected from the group consisting of: (a) a non-hydrophilic active agent and (b) a hydrophilic active agent dissolved or suspended in an oil;
wherein the shell comprises a polymeric coating, and wherein at least a portion of the microcapsules in a sample of the composition are spherical when the sample of the microcapsules is viewed in a scanning electron microscope with a magnification in the range of between ×2000 and ×50000.

17. The method of claim 16, wherein the physiologically acceptable carrier is suitable for oral administration or suitable for administration by injection.

18. The method of claim 16, wherein the delivery of the active agent is via the intestinal paracellular, trancellular or lymphatic transport, or a combination thereof.
